(19) United States
(12) Patent Application Publication
(10) Pub. No.: US 2008/0009540 A1
(43) Pub. Date: Jan. 10, 2008

(54) Amorphous Atorvastatin Calcium

(76) Inventors: Srinivasulu Gudipati, Hyderabad (IN); Srinivas Katkam, Secundarabad (IN); Satyanarayana Komati, Hyderabad (IN); Satyanarayana Jaya Kudavalli, West Godhavari (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/576,396

(22) PCT Filed: Sep. 29, 2005
(86) PCT No.: PCT/US05/35094

§ 371 Date: Mar. 30, 2007

Related U.S. Application Data
(60) Provisional application No. 60/614,578, filed on Sep. 30, 2004.

(30) Foreign Application Priority Data
Jun. 10, 2005 (IN) 715/CHE/2005

(51) Int. Cl. A61K 31/401; A61P 43/00

(52) U.S. Cl. 514/423

(57) ABSTRACT

Amorphous atorvastatin calcium having an enhanced stability contains about 2 to about 8 percent by weight water. A process for preparing the amorphous atorvastatin calcium and a packaging system for maintaining the stability are described.

### INTRODUCTION TO THE INVENTION

The invention relates to a stable amorphous form of atorvastatin calcium and to packaging procedures that maintain stability of the compound during storage.

Atorvastatin calcium is a drug compound that is used as a lipid-lowering agent, for treating hypercholestrolemia. The compound has the chemical name [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1). Pharmaceutical products containing crystalline atorvastatin calcium trihydrate are sold using the trademark LIPITOR.

Atorvastatin calcium exists in various crystalline and amorphous forms. The amorphous form is of interest, due at least in part to its enhanced solubility as compared to crystalline forms, a higher solubility thought to provide an improved bioavailability profile. A process for preparing amorphous atorvastatin calcium from crystalline form I of the compound is described in International Patent Publication WO 97/03960, which states that prior processes had prepared the amorphous compound but, after the advent of crystalline forms, there was a problem with producing the amorphous material reproducibly. A number of subsequent patents and applications relate to different processes for producing amorphous atorvastatin calcium.

International Patent Publication WO 2004/032920 describes the poor storage stability of atorvastatin calcium, and states that the compound is susceptible to adverse effects from heat, light, moisture, and low pH conditions. The amorphous form of a compound was said to be more susceptible to these adverse conditions. As described in this publication, formulated amorphous atorvastatin calcium solid dosage forms were protected against degradation by storage in an inert atmosphere, protected from the external environment by a layer that is not permeable to gases. In one example, tablets containing the drug were packaged in aluminum foil blisters, the blisters having an argon atmosphere.

There remains a need to provide amorphous atorvastatin calcium on a commercial scale, the product having enhanced stability.

### SUMMARY OF THE INVENTION

In one aspect, the invention includes amorphous atorvastatin calcium having a water content about 2 to about 8 percent by weight.

In another aspect, the invention includes amorphous atorvastatin calcium having a water content about 2 to about 8 percent by weight, and having a total organic compound impurity content less than about 1 area-percent by high performance liquid chromatography.

In yet another aspect, the invention includes amorphous atorvastatin calcium having a water content about 2 to about 8 percent by weight, and further comprising a pharmaceutical excipient or additive. an example of a useful additive being an antioxidant, such as about 0.05 to about 0.2 percent by weight of butylated hydroxyanisole, butylated hydroxytoluene, propyl gallate, or a mixture of two or more thereof.

In yet another aspect, the invention includes amorphous atorvastatin calcium having a water content about 2 to about 8 percent by weight, being packaged in a first closed container that is substantially non-permeable to moisture, the first container being disposed within a second closed container that is substantially non-permeable to moisture, and space between the containers containing at least one of an inert gas, a desiccant, and an oxygen absorber.

In a further aspect, the invention includes amorphous atorvastatin calcium having a water content about 2 to about 8 percent by weight, being packaged in a first closed container that is substantially non-permeable to moisture, the first container being disposed within a second closed container that is substantially non-permeable to moisture, and the second container being disposed within a third closed container that is substantially non-permeable to moisture, and space between any two containers containing at least one of an inert gas, a desiccant, and an oxygen absorber.

Another aspect of the invention includes amorphous atorvastatin calcium having a water content about 2 to about 8 percent by weight, prepared by a process comprising:

(a) dissolving atorvastatin calcium in an ester solvent;

(b) removing the solvent to form an amorphous solid; and

(c) exposing the amorphous solid to a humidified atmosphere.

### BRIEF DESCRIPTION OF THE DRAWINGS

FIG. 1 is an X-ray diffraction pattern for the amorphous atorvastatin calcium product of Example 4.

FIG. 2 is a schematic representation of a process for preparing atorvastatin calcium.

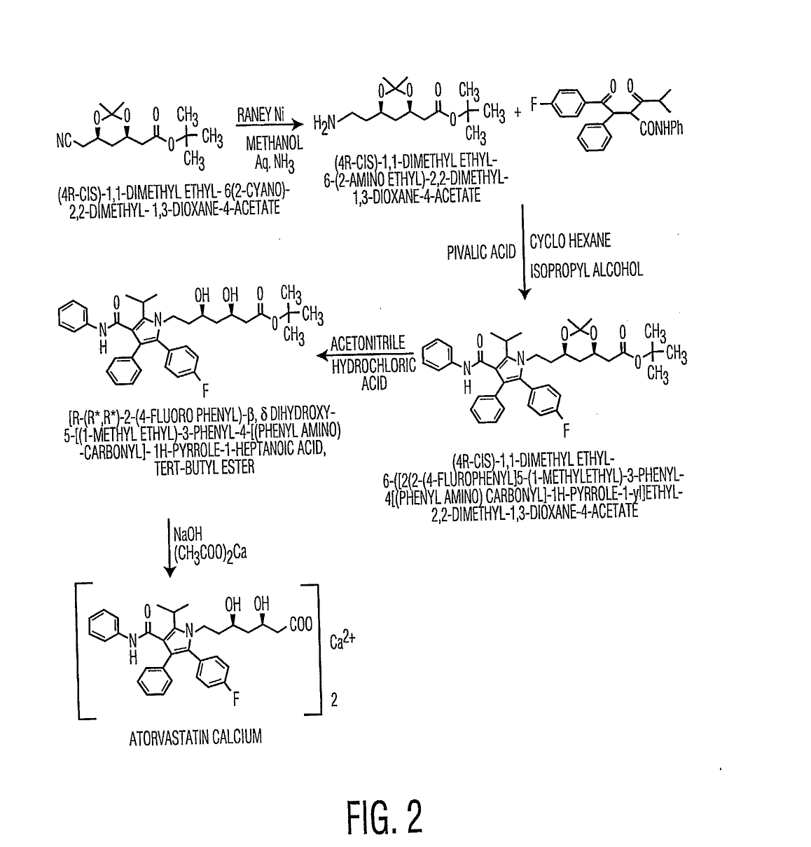

### DETAILED DESCRIPTION

The present invention is based on a finding that amorphous atorvastatin calcium having a moisture content about 2 to about 8 percent by weight has an enhanced stablility during storage. One aspect of the invention, therefore, is an amorphous atorvastatin calcium having this moisture content.

Another aspect of the invention is a process for preparing amorphous atorvastatin calcium having a moisture content about 2 to about 8 percent by weight.

An embodiment of a process for the preparation of a stable amorphous atorvastatin calcium having moisture content about 2-8 percent w/w comprises the steps of:

i. dissolving atorvastatin calcium in an ester solvent, optionally adding a pharmaceutical excipient or additive;

ii. removing the solvent to produce an amorphous residue;

iii. optionally micronizing the residue to produce a desired particle size distribution, optionally followed by drying; and

iv. exposing the residue to a humidified atmosphere to produce the stable amorphous atorvastatin calcium having a moisture content of about 2 to about 8 percent by weight.

The process of the present invention involves dissolving atorvastatin calcium in an ester solvent, with or without the addition of pharmaceutical excipients or additives. Useful ester solvents include but are not limited to ethyl acetate, t-butyl acetate, propyl acetate, and the like. The concentration of atorvastatin calcium in the solution will frequently range from 6 to 9% w/v, but the process can be successfully conducted using other concentrations.

The dissolution temperature frequently will range from 50 to 85° C. Suitable temperatures for particular atorvastatin calcium-solvent mixtures will vary for different solvents due to the variations in atorvastatin solubility in individual solvents. The temperature will be chosen to obtain a clear solution having the desired solute concentration.

In one embodiment, one or more pharmaceutical excipients or additives, or mixtures thereof, can be dissolved in the solution, or solutions of the excipients or additives can be mixed with the atorvastatin calcium solution. The pharmaceutical excipients or additives that can be used include but are not limited to antioxidants, surfactants, dispersants, lubricants, and other stabilizing additives. Examples of suitable antioxidants include but are not limited to butylated hydroxyanisole, butylated hydroxytoluene, propyl gallate and the like; including these substances can provide additional storage stability for the amorphous atorvastatin calcium.

The concentration of antioxidant in the final atorvastatin calcium product can be about 0.05 to about 0.2 percent by weight, and will frequently be less than about 0.15 percent by weight, while concentrations of other excipients and additives can vary widely. The pharmaceutical excipient or additive can be added to the solution of ester solvent containing atorvastatin calcium at temperatures that will not affect the stability of the excipient or additive, with mixing sufficient to produce a homogenous solution.

The solvent can be removed from the solution by any desired technique, such as agitated thin film drying, spray drying, rotational evaporation (such as using a Buchi Rotavapor), spin-flash drying, fluid-bed drying, lyophilization or other techniques known to those skilled in the art.

When an agitated thin film dryer is used, a temperature of the apparatus from about 50 to about 120° C. has been found useful, for drying conducted under a vacuum less than about 650 mm Hg. The temperature and vacuum conditions can be varied depending upon the solvent used and the characteristics of the apparatus.

After the solvent has been removed, it frequently is desired to reduce the particle sizes of the residue, since the subsequent production of dosage forms such as tablets generally requires fine particle sizes. In certain embodiments, the desired particle size distribution is expressed by D₉₀<20 μm, meaning that 90 volume percent of the particles have diameters less than 20 μm. In certain instances a desired particle size distribution is D₉₀<15 μm. Such particle size information can be obtained using a Malvern laser light scattering particle size analyzer, sold by Malvern Instruments Ltd. of Worcestershire, United Kingdom. Many techniques are useful for particle size reduction, including jet milling. In an embodiment, the residue is micronized in a jet mill using gases such as air or nitrogen at pressures about 4.5 to 6 kg/cm².

The micronized amorphous atorvastatin calcium can optionally be dried to remove residual solvent using a suitable drying process, such as tray drying, fluid bed drying, microwave drying, belt drying, rotary drying, aerial drying, oven drying and other drying processes known in the art, with or without application of vacuum and/or under inert conditions. In one embodiment the micronized amorphous atorvastatin calcium is dried at a temperature between about 25 to 80° C. over a period of 4 to 7 hours, to produce a residual solvent content less than about 5000 ppm by gas chromatography. Of course, the temperatures and times for drying will be chosen based on the properties of the solvent to be removed, and must be selected to not affect the amorphous atorvastatin calcium itself. Some solvent removal techniques will produce a residue already having a low solvent content, so that further drying will not be required.

The amorphous atorvastatin calcium is exposed to a humidified atmosphere, such as air or an inert gas. The relative humidity in the humidified atmosphere typically ranges from about 60 to about 90% to afford the final desired moisture content. Exposure will be continued for a time sufficient to produce the moisture content of the final amorphous atorvastatin calcium between about 2 to about 8 percent by weight, and that time will vary depending upon the mode of exposure and the moisture content of the atmosphere. Determination of an appropriate time is a matter of simple experimentation.

Drying and exposure to humidity does not need to be performed sequentially. In an embodiment of the invention, the drying operation is performed by passing humidified gas, such as air or nitrogen, through or over the amorphous atorvastatin calcium, at temperatures suitable for removing the solvent.

A further aspect of the present invention is providing a packaging environment for the stable amorphous atorvastatin calcium having the moisture content about 2-8 percent w/w, which is useful on a commercial scale and preserves the moisture and impurity content.

The packaging environment for the storage of the stable amorphous atorvastatin calcium of this invention comprises packaging amorphous atorvastatin calcium with a moisture content of about 2 to 8 percent w/w into non-permeable containers with or without an oxygen absorbent, with or without an inert gas environment, and with or without a desiccant.

In one embodiment, stability of amorphous atorvastatin calcium is maintained during storage by a procedure comprising packaging amorphous atorvastatin calcium having a moisture content of about 2 to 8 percent w/w into a non-moisture permeable container with or without inert gas, followed by placing into a second non-moisture permeable container containing a desiccant pouch, with or without an oxygen absorbent, inert gas, or both, and further placing the second container into a non-moisture permeable container containing a desiccant and an inert gas.

Non-moisture permeable containers include but are not limited to polyethylene or other polymer bags, including laminated bags, glass containers, metal containers, rigid plastic containers and the like. The polymer bags can be clear or transparent, or have colors such as black or white.

Useful inert gases include, but are not limited to, nitrogen and argon. Useful oxygen absorbers include, but are not limited to, powdered iron compositions and compositions containing unsaturated organic compounds; several types of oxygen absorber products are commercially available, including a product that absorbs both moisture and oxygen. Suitable desiccants include chemically inert substances such as dried silica gel or a zeolite. The desiccants and oxygen absorbers will be contained in forms such as pouches or bags that are permeable to gases, such a cloth bags, paper bags, or porous plastic containers.

The field of oxygen absorbers is evolving, as shown by recent patent documents. U.S. Pat. No. 6,596,192 B2 to Himeshima et al. describes a dual oxygen-moisture absorbing composition containing a liquid unsaturated hydrocarbon, a transition metal-organic compound, and an inorganic compound such as calcium oxide, calcium chloride, or magnesium chloride. Published Patent Application Publication US 2005/0034599 A1 describes an oxygen absorbing composition containing powdered iron that is coated with an oxidation-promoting alkali metal or alkaline earth metal salt. Any of the older and newer oxygen absorbing compositions will be useful in the present invention.

In an embodiment, storage-stable amorphous atorvastatin calcium is achieved by the procedure of placing amorphous atorvastatin calcium having a moisture content of about 2 to 8 percent w/w into a non-permeable container such as a polyethylene bag with or without an inert gas, followed by placing the container into a second non-permeable container such as a polyethylene bag optionally containing a desiccant pouch, with or without an oxygen absorbent, an inert gas, or both, then further placing the second container into a third non-permeable container such as a triple laminated polyethylene-aluminum foil bag containing a desiccant pouch and purged with an inert gas, then finally placing the triple laminated bag into a high density polyethylene storage container (HDPE) and sealing the container. Typically, the bags will each be sealed, such as using a tie or adhesive, heat welding, etc.

In another embodiment, 5 Kg of amorphous atorvastatin calcium having a moisture content between about 2 and about 8 percent by weight are placed into a first polyethylene bag, the bag is purged with nitrogen, excess nitrogen is removed, and the bag is sealed. The first bag is then placed into a second polyethylene bag, the second bag is purged with nitrogen, oxygen absorber pouches (having the trademark AGELESS, manufactured by Mitsubishi Gas Chemical Company, Tokyo, Japan and rated for absorbing 10 liters of oxygen) and a 100 gram silica gel desiccant pouch are added, and the second bag is sealed. The second bag is placed into a triple laminated bag containing a 100 gram silica gel desiccant pouch, then the triple laminated bag is purged with nitrogen and heat sealed. For shipping, one or more triple laminated bags will be placed into a sealed container having adequate physical strength, such as a high density polyethylene drum.

Depending on the actual sizes of oxygen absorbers and desiccant packages used, more than one package could be required to obtain the desired quantities. For example, 100 grams of silica gel can be provided in the form of four 25 gram pouches.

The stable amorphous atorvastatin calcium of the present invention is well suited for pharmaceutical formulations. In general, amorphous atorvastatin calcium for use in pharmaceutical formulations is desired to have a total organic impurity content no greater than about 1 area-percent, as determined using high performance liquid chromatography.

The present processes for the preparation and packaging of stable amorphous atorvastatin calcium are simple, cost-effective, and non-hazardous.

The following examples illustrate certain aspects of the invention in greater detail and are not to be construed as limiting any aspect of the invention.

### EXAMPLE 1

### Preparation of (4R-Cis)-1,1-dimethylethyl-6-(2-amino Ethyl)-2,2-dimethyl-1,3-dioxane-4-acetate

75 ml of methanol was charged into a hydrogenation vessel at 0-5° C. 15 grams of Raney nickel was washed under a nitrogen atmosphere with 100 ml of water and added to the hydrogenation vessel at the same temperature. 14.3 ml of aqueous 15-18% ammonia was added, then 20.0 grams of (4R,Cis)-1,1-dimethylethyl-6-(2-cyano)-2,2-dimethyl-1,3-dioxane-4-acetate was dissolved in 45 ml of methanol and added to the vessel. The vessel was maintained under a hydrogen pressure of 3 to 3.5 Kg/cm² at 30-40° C. for 6-8 hours. After completion of the reaction, the reaction mass was filtered through a flux calcined diatomaceous earth bed and washed with 36 ml of methanol. The solvent was distilled from the filtrate under vacuum at temperatures below 60° C., then 52 ml of methanol were added and again the solvent was distilled, and this was repeated two additional times with 52 ml portions of methanol. Finally, the solvent was distilled completely to produce 18±2 grams of the title compound having a moisture content by the Karl Fischer method not more than about 5% by weight and a purity of at least about 95% by high performance liquid chromatography.

### EXAMPLE 2

### Preparation of (4R-Cis)-1,1-dimethylethyl-6[2[-(4-fluorophenyl]-5-(1-methylethyl)-3-phenyl-4-[(Phenylamino) Carbonyl]-1H-pyrrole-1YL]ethyl]-2,2-dimethyl-1,3-dioxane-4-acetate

80.0 grams of (4R-cis)-1,1-dimethylethyl-6-(2-aminoethyl)-2,2-dimethyl-1,3-dioxane-4-acetate, 1200 ml of cyclohexane and 104 grams of (±)-4-fluoro-α-2-methyl-1-oxopropyl-γ-oxo-N-β-diphenyl benzene were stirred at 25-30° C. for 15-20 minutes. 16 grams of pivalic acid were added, and then the reaction mass was heated to reflux under azeotropic conditions until completion of the reaction. The solvent was evaporated below 70° C. under reduced pressure. Isopropyl alcohol (160 ml) was added and evaporated under reduced pressure. 400 ml of isopropyl alcohol was added to the residue below 50° C., then the mixture was cooled to 25-30° C. for about 6-8 hours. The mixture was further cooled to 0-10° C. and stirred for 2-3 hours. The separated solid was filtered and washed with 160 ml of isopropyl alcohol. The obtained solid was dried at 60-70° C. to yield 120 grams of the title compound.

### EXAMPLE 3

### Preparation of [R-(R*,R*)]-2-(4-fluorophenyl)β,δ-dihydroxy-5-(1-methylethyl)-3-phenyl-4-[phenyl amino) carbonyl]-1H-pyrrole-1-Heptanoic Acid Tertiary Butyl Ester

110.0 grams of (4R-cis)-1,1-dimethylethyl-6[2[-(4-fluorophenyl]-5-(1-methylethyl)-3-phenyl-4-[(phenylamino)carbonyl]-1H-pyrrole-1yl]ethyl]-2,2-dimethyl-1,3-dioxane-4-acetate was dissolved in 1100 ml of acetonitrile at 30-35° C. A solution of concentrated hydrochloric acid (44 ml) in water (396 ml) was added to the reaction mass at 25-30° C. over about 30-45 minutes. After stirring the contents at 25-30° C. until the completion of the reaction, the reaction mass was diluted with 1100 ml of water. The pH of the reaction mass was adjusted to 7-8 by adding a solution of sodium carbonate (31.6 grams) in water (158 ml) at 10-15° C. The formed solid was filtered and washed with 550 ml of water. Wet solid was dissolved in a mixture of 924 ml of acetonitrile and 365 ml of water at 70-75° C. The solution was cooled to 25-30° C. for about 1-2 hours, and the precipitated solid was filtered and washed with a mixture of 146 ml of acetonitrile and 73 ml of water. The washed solid was dried at 50-60° C. for about 8-12 hours to yield 85 grams of the title compound.

### EXAMPLE 4

### Preparation of [R-(R*, R*) 2-(4-fluorophenyl)-β,δ-dihydroxy-5-(1-methylethyl)-3-phenyl-4-[(Phenylamino)Carbonyl)-1H-Pyrrole-1-Heptanoicacid Hemicalcium Salt (Amorphous Atorvasatin Calcium)

200.0 grams of R—(R*,R*)]-2-(4-fluorophenyl)β,δ-dihydroxy-5-(1-methylethyl)-3-phenyl-4-[phenylamino)carbonyl]-1H-pyrrole-1-heptanoic acid tertiary butyl ester, 500 ml of water, 14.3 grams of sodium hydroxide flakes and 1600 ml of acetonitrile were heated to 40-45° C. for about 2 hours. A solution of calcium acetate (31.8 grams) in water (318 ml) was added to the reaction mass slowly over 30-60 minutes at 40-45° C. A solution of sodium hydroxide (1 gram) in water (10 ml) was further added, and the temperature of the reaction mass was raised to 70-80° C. for about 1 hour. The reaction mass was filtered on a flux calcined diatomaceous earth bed at 60-65° C. and washed with acetonitrile (200 ml). 2400 ml of water were added to the filtrate at 25-30° C. followed by addition of a solution of sodium hydroxide (0.4 grams) in water (4 ml) and heating to 70-80° C. for 6-8 hours. The reaction mass was cooled to 25-30° C. for about 2 hours, and the obtained solid was filtered and washed with water (500 ml). Then the solid was dried until its moisture content was less than about 2% by weight. The solid was dissolved in 1600 ml of ethyl acetate at 60-70° C., and the solution was then cooled immediately to 25-35° C. The organic layer was processed through an agitated thin film dryer at 68-78° C. under reduced pressure and the solid amorphous output material was micronized to a particle size distribution having 90 volume percent of the particles with diameters less than 15 μm. The material was dried in a fluidized bed dryer at 60 to 75° C. for 4 hours and then humidified air (70-80% relative humidity) was passed over the solid for 2 to 4 hours to produce 100 grams of amorphous atorvastatin calcium having a moisture content of 5.4 percent by weight by the Karl Fischer method, with a 99.6% organic compound purity by high performance liquid chromatography (“HPLC”).

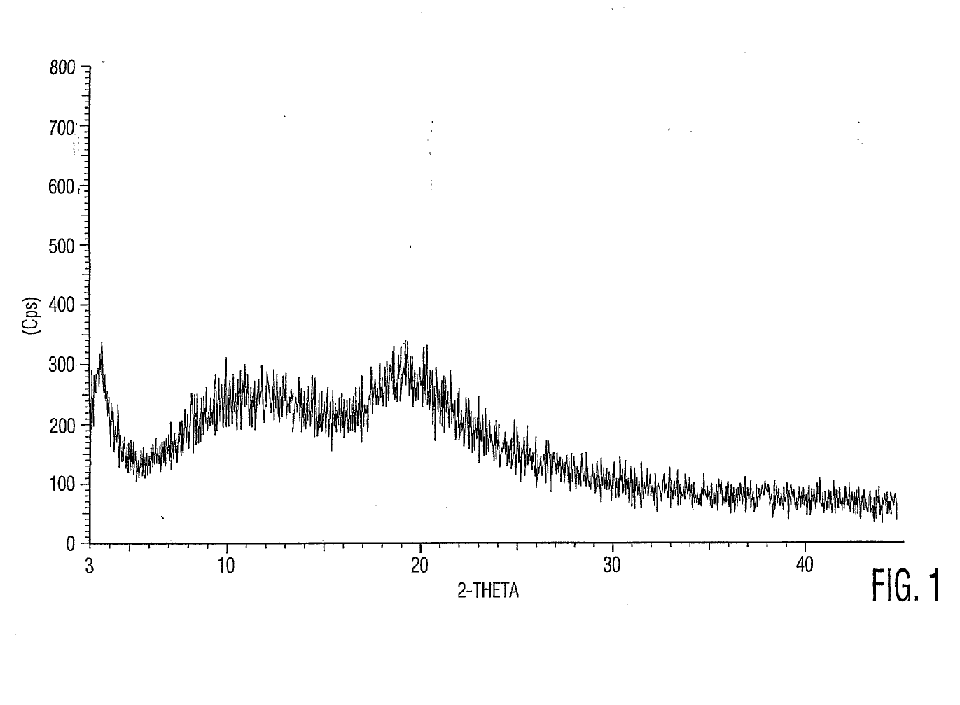

The X-ray diffraction pattern from a sample of the product is shown as FIG. 1, where the y-axis is intensity and the x-axis is the 2θ angle, in degrees. This pattern was obtained using Cu Kα radiation (1.541 Å).

### EXAMPLE 5

### Stabilization of Amorphous Atorvastatin Calcium with Butylated Hydroxyanisole

160 g of atorvastatin calcium was added to 1600 ml of ethyl acetate followed by heating to a temperature of about 65 to 75° C. to obtain a clear solution, then the clear solution was cooled to a temperature of about 25 to 30° C. 0.2 g of butylated hydroxyanisole were added to the above solution followed by stirring for about 5 to 10 minutes. The organic layer was filtered through celite followed by washing with 160 ml of ethyl acetate and the organic layer was passed through an agitated thin film drier at a temperature of about 73 to 78° C. by applying vacuum of about 650 mm Hg. The solid material that was obtained from the agitated thin film drier was subjected to micronization in a jet mill to produce a solid material having a particle size distribution of D₉₀<15 μm. The solid material was then dried using a fluid bed drier at a temperature of about 68 to 75° C. for 4 hours with simultaneous passing of humidified air having a relative humidity of 70 to 80% to yield a stable amorphous atorvastatin calcium having a moisture content of 3 to 8 percent w/w. (Yield: 80%).

Physical characteristics of the micronized product were as follows:

| Particle Size by Malvern | | | Bulk Density, g/cm³ | |
|---|---|---|---|---|
| D₁₀ | D₅₀ | D₉₀ | Before Tapping | After Tapping |
| <0.6 μm | <1.89 μm | <4.29 μm | 0.29 | 0.47 |

### EXAMPLE 6

The following packaging procedure was followed: 1) amorphous atorvastatin calcium having a 6 percent w/w water content was initially packed in a polyethylene bag, which was then tied; 2) the tied polyethylene bag was placed in a black polyethylene bag with a silica gel pouch under a nitrogen atmosphere and then sealed; 3) the sealed black polyethylene bag was placed in a triple laminated bag along with a silica gel pouch, was purged with nitrogen, and sealed; and 4) the sealed laminated bag was stored in a HDPE drum.

Stability of the amorphous atorvastatin calcium during storage at 40° C. and 75% relative humidity is shown in the following table, where water was determined by the Karl Fischer method, and total organic impurities are expressed as area-percent, as determined by high performance liquid chromatography.

| Time | Wt. Percent H₂O | Total Impurities |
|---|---|---|
| Initial | 6.00 | 0.37 |
| 45 Days | 3.49 | 0.61 |
| 2 Months | 3.47 | 0.50 |

Stability of the amorphous atorvastatin calcium during storage at 25° C. and 60% relative humidity is shown in the following table.

| Time | Wt. Percent H₂O | Total Impurities |
|---|---|---|
| Initial | 6.00 | 0.37 |
| 45 Days | 3.70 | 0.51 |
| 2 Months | 3.97 | 0.49 |

Stability data of the amorphous atorvastatin calcium during storage at 2 to 8° C. is shown in the following table.

| Time | Wt. Percent H₂O | Total Impurities |
|---|---|---|
| Initial | 6.00 | 0.37 |
| 45 Days | 4.50 | 0.46 |
| 2 Months | 4.80 | 0.47 |

These data indicate that the amorphous atorvastatin calcium did not change appreciably during the storage periods.

### EXAMPLE 7

The following packaging procedure was followed: 1) amorphous atorvastatin calcium having a 2 percent w/w moisture content was placed in a white polyethylene bag, the bag was purged with nitrogen, and then the bag was tied; 2) the tied white polyethylene bag was placed into a black polyethylene bag, along with a silica gel pouch and and a bag of oxygen absorbent, then the black bag was purged with nitrogen and sealed; 3) the sealed black polyethylene bag was placed into a triple laminated bag along with a silica gel pouch, and then the laminated bag was purged with nitrogen and sealed; and 4) the sealed laminated bag was stored in a HDPE drum.

Stability of the amorphous atorvastatin calcium during storage at 40° C. and 75% relative humidity is shown in the following table.

| Time | Wt. Percent H₂O | Total Impurities |
|---|---|---|
| Initial | 2.0 | 0.29 |
| 15 Days | 3.1 | 0.34 |
| 1 Month | 3.4 | 0.41 |
| 45 Days | 3.2 | 0.34 |
| 2 Months | 3.2 | 0.40 |

Stability of the amorphous atorvastatin calcium during storage at 25° C. and 60% relative humidity is shown in the following table.

| Time | Wt. Percent H₂O | Total Impurities |
|---|---|---|
| Initial | 2.0 | 0.29 |
| 1 Month | 2.4 | 0.34 |
| 2 Months | 2.9 | 0.38 |

Stability of the amorphous atorvastatin calcium during storage at 2 to 8° C. is shown in the following table.

| Time | Wt. Percent H₂O | Total Impurities |
|---|---|---|
| Initial | 2.0 | 0.29 |
| 1 Month | 2.3 | 0.27 |
| 2 Months | 2.6 | 0.30 |

These data indicate that the amorphous atorvastatin calcium did not change appreciably during the storage periods.

### EXAMPLE 8

A commercial-sized batch of amorphous atorvastatin calcium was packaged as follows:

a) amorphous atorvastatin calcium having a 6 percent w/w water content was initially placed into a clear polyethylene bag, air was removed, and the clear polyethylene bag was tied;

b) the tied clear polyethylene bag was placed into a black polyethylene bag along with a silica gel pouch and a package of oxygen absorbent, and then the black polyethylene bag was purged with nitrogen and sealed; and

c) the sealed black polyethylene bag was placed in a triple laminated bag along with a silica gel pouch, then the laminated bag was purged with nitrogen and sealed.

Stability of the amorphous atorvastatin calcium during storage at 40° C. and 75% relative humidity is shown in the following table.

| Time | Wt. Percent H₂O | Total Impurities |
|---|---|---|
| Initial | 5.4 | 0.35 |
| 15 Days | 4.8 | 0.39 |
| 1 Month | 4.3 | 0.45 |
| 45 Days | 4.3 | 0.45 |
| 2 Month | 4.5 | 0.47 |
| 3 Month | 5.0 | 0.48 |

Stability of the amorphous atorvastatin calcium during storage at 25° C. and 60% relative humidity is shown in the following table.

| Time | Wt. Percent H₂O | Total Impurities |
|---|---|---|
| Initial | 5.4 | 0.35 |
| 1 Month | 4.6 | 0.41 |
| 2 Month | 4.8 | 0.42 |
| 3 Month | 4.9 | 0.40 |

Stability of the amorphous atorvastatin calcium during storage at 2 to 8° C. is shown in the following table.

| Time | Wt. Percent H₂O | Total Impurities |
|---|---|---|
| Initial | 5.4 | 0.35 |
| 1 Month | 5.4 | 0.37 |
| 2 Month | 5.3 | 0.36 |
| 3 Month | 5.4 | 0.34 |

These data indicate that the amorphous atorvastatin calcium did not change appreciably during the storage periods.

1. Amorphous atorvastatin calcium having a water content about 2 to about 8 percent by weight.

2. The amorphous atorvastatin calcium of claim 1, having a water content about 4 to about 7 percent by weight.

3. The amorphous atorvastatin calcium of claim 1, having a water content about 3 to about 6 percent by weight.

4. The amorphous atorvastatin calcium of claim 1, having a water content about 4 to about 6 percent by weight.

5. The amorphous atorvastatin calcium of claim 1, prepared by a process comprising:
(a) dissolving atorvastatin calcium in an ester solvent;
(b) removing the solvent to form an amorphous solid; and
(c) exposing the amorphous solid to a humidified atmosphere.

6. The amorphous atorvastatin of claim 5, wherein the ester solvent of (a) comprises ethyl acetate.

7. The amorphous atorvastatin calcium of claim 5, wherein solvent removal in (b) is conducted by agitated thin film drying, spray drying, rotational evaporation, spin-flash drying, fluid-bed drying, or lyophilization.

8. The amorphous atorvastatin calcium of claim 5, wherein solvent removal in (b) is conducted by agitated thin film drying.

9. The amorphous atorvastatin calcium of claim 5, wherein the humidified atmosphere of (c) comprises air having about 60 to about 90 percent relative humidity.

10. The amorphous atorvastatin calcium of claim 5, wherein the humidified atmosphere of (c) is at a temperature about 60 to about 80° C.

11. The amorphous atorvastatin calcium of claim 1, further comprising a pharmaceutical excipient or additive.

12. The amorphous atorvastatin calcium of claim 1, further comprising an antioxidant additive.

13. The amorphous atorvastatin calcium of claim 1, further comprising butylated hydroxyanisole, butylated hydroxytoluene, propyl gallate, or a mixture of two or more thereof.

14. The amorphous atorvastatin calcium of claim 1, further comprising about 0.05 to about 0.2 percent by weight of butylated hydroxyanisole, butylated hydroxytoluene, propyl gallate, or a mixture of two or more thereof.

15. The amorphous atorvastatin calcium of claim 1, having a total organic compound impurity content less than about 1 area-percent by high performance liquid chromatography.

16. The amorphous atorvastatin calcium of claim 15, being packaged in packaging comprising a first closed container that is substantially non-permeable to moisture, the first container being disposed within a second closed container that is substantially non-permeable to moisture, and space between the containers being provided with at least one of an inert gas, a desiccant, and an oxygen absorber.

17. The amorphous atorvastatin calcium of claim 15, being packaged in packaging comprising a first closed container that is substantially non-permeable to moisture, the first container being disposed within a second closed container that is substantially non-permeable to moisture, and the second container being disposed within a third closed container that is substantially non-permeable to moisture, and space between any two containers being provided with at least one of an inert gas, a desiccant, and an oxygen absorber.

18. The amorphous atorvastatin calcium of claim 17, wherein a space between two containers is provided with more than one of an inert gas, a desiccant, and an oxygen absorber.

19. The amorphous atorvastatin calcium of claim 17, wherein a space between two containers is provided with an oxygen absorber.

20. The amorphous atorvastatin calcium of claim 17, wherein a container comprises a polymeric bag.
